(19) United States
(12) Plant Patent Application Publication
(10) Pub. No.: US 2002/0129411 P1
(43) Pub. Date: Sep. 12, 2002

(54) Hybrid sarracenia pitcher plant named 'Redbug'

(76) Inventors: T. Lawrence Mellichamp, Charlotte, NC (US); Robert K. Gardner, Chapel Hill, NC (US)

Correspondence Address: MYERS BIGEL SIBLEY & SAJOVEC, PO BOX 37428, RALEIGH, NC, 27627, US

(21) Appl. No.: 09/755,823

(22) Filed: Jan. 5, 2001

(51) Int. Cl. A01H 5/00

(52) U.S. Cl. PLT/263

(57) ABSTRACT

A new and distinct cultivar of Sarracenia called ‘Redbug’ is described that is a vigorously growing, compact plant having dark Chrysanthemum Crimson (H.C.C. #860/2) pitchers, suffused with veins of a deeper cast of Chrysanthemum Crimson. The pitcher is distinguished from other Sarracenia by having a higher width-to-length ratio, a wider hood, and a prominent horizontal filament. Furthermore, in the winter the leaves retain color and texture and are hardy to United States Department of Agriculture Hardiness Zone 6.

### LATIN NAME OF THE GENUS AND SPECIES

The Latin name of the novel hybrid plant variety disclosed herein is Sarracenia rubra ssp. wherryi×Sarracenia rubra ssp. rubra.

### VARIETY DENOMINATION

The inventive cultivar of Sarracenia disclosed herein has been given the variety denomination ‘Redbug’.

### BACKGROUND OF THE INVENTION

Sarracenia species are long-lived, herbaceous, perennial, insectivorous plants found growing in open, sunny, moist meadows or long-leaf pine savannas predominantly found throughout the southeastern United States. The plants produce pitchers which are hollow, modified leaves. Each pitcher has a hood, which typically covers the mouth of the pitcher to keep rainwater out and insect prey trapped inside. Insects are attracted by the color of the pitchers, the nectar produced around the mouth of the pitcher, and the smell of decaying insects within the pitcher. The ability to absorb nutrients from digested insect prey has been shown to supply necessary nourishment to the roots, stems, and leaves of Sarracenia spp.; and may account for the ability of these plants to thrive predominantly in nutrient-poor acidic soils characteristic of the southeastern United States.

Sarracenia pitcher plants have been of horticultural interest for several hundred years. One problem in the production of commercially-available Sarracenia pitcher plants is the lack of horticulturally superior plants. To meet the current demand, wild species are being harvested, resulting in a depletion of native populations. There are approximately eight species found in the wild, and these species can cross-pollinate to produce fertile hybrids that grow well in cultivation. Therefore, it would be desirable to produce new, more robust cultivars of pitcher plants with enhanced characteristics such as compact growth, colorful leaves, and winter foliage to meet the current horticultural demand.

### BRIEF SUMMARY OF THE INVENTION

The present Sarracenia ‘Redbug’ hybrid plant (Sarracenia rubra ssp. wherryi×Sarracenia rubra ssp. rubra) is a dwarf cultivar with inflated pitchers toward the apex of the leaf. The plant has dark Chrysanthemum Crimson (Horticultural Color Chart (H.C.C.) #860/2, page 175) tubular leaves, suffused with veins of a deeper cast of Chrysanthemum Crimson (H.C.C. #860/2, page 175). The pitcher leaves are thick, have very fine but distinct fuzz covering the entire surface, have a width-length ratio of 0. 1, and prolonged retention of foliage with attractive leaf color during the dormant season. The hood of Sarracenia ‘Redbug’ completely covers and extends beyond the leaf orifice and often has a slightly scalloped or wavy margin, and a prominent horizontal filament up to 2 mm long. Furthermore, this plant is a vigorous, compact grower (short in stature with many pitchers), forming a clump from a single crown in one growing season with flushes of new pitchers being produced all summer.

Lineage.—The ‘Redbug’ cultivar originated as a single hybrid seedling at the North Carolina Botanical Garden, part of the University of North Carolina at Chapel Hill, in the late 1980s. ‘Redbug’ is a hybrid derived from a cross of Sarracenia rubra subspecies rubra (a native dwarf pitcher plant from coastal North Carolina) and Sarracenia rubra subspecies wherryi, a native species from southeastern Alabama. In 1990, the single individual plant now known as ‘Redbug’ was selected because of its multitude of exceptional features, and has been propagated asexually since that time. While each parental species had only a subset of horticulturally desirable features, ‘Redbug’ attained the full composite of features including vibrant color, compact growth, and winter foliage retention. It showed exceptional hybrid vigor and grew well under a variety of conditions.

Asexual Reproduction.—Sarracenia ‘Redbug’ was first asexually reproduced at the North Carolina Botanical Garden in Chapel Hill, North Carolina by division of the rhizome, i.e., clump division, in 1990. The combination of characteristics disclosed herein for Sarracenia ‘Redbug’ is stable through successive generations of asexual reproduction.

### BRIEF DESCRIPTION OF THE DRAWINGS

FIG. 1 is a color photograph of a typical specimen of the Sarracenia ‘Redbug’ hybrid in the summer receiving full sunlight. Leaves at various stages of development are presented.

### DETAILED BOTANICAL DESCRIPTION

The following is a detailed description of the botanical characteristics of a new and distinct cultivar of Sarracenia plant known by the cultivar name ‘Redbug’, and botanically known as Sarracenia rubra ssp. wherryi×Sarracenia rubra ssp. rubra. All colors cited herein refer to the Horticultural Colour Chart (H.C.C.) designations, issued by The British Colour Council in collaboration with The Royal Horticultural Society. Where dimensions, sizes, colors, and other characteristics are given, it is to be understood that such characteristics are approximations or averages set forth as accurately as practicable. The descriptions reported herein are from specimens grown outdoors in full sunlight in Chapel Hill, N.C. and Charlotte, N.C. unless otherwise noted. ‘Redbug’ has not been observed under all possible environmental conditions. The phenotype may vary with variations in the environment such as temperature, light intensity, day length, cultural conditions, and the like.

Growth Conditions.—Sarracenia ‘Redbug’ is a strong grower and adaptable to pot culture. It prefers 100% full sunlight and warm temperatures to bring out the deepest cast of Chrysanthemum Crimson (H.C.C. #860/2, page 175). While new pitcher leaves may be greenish at first, they become a dark-red color as the leaves develop into mature pitchers (within 3-4 weeks). This trait is most similar to the S. rubra ssp. rubra parent. ‘Redbug’ prefers an acidic, peat-based, constantly-moist soil.

During the winter (or under similar day length and temperature conditions as experienced during winter), there is a prolonged retention of foliage, and the leaves maintain color and a fuzzy texture. ‘Redbug’ is hardy to United States Department of Agriculture Hardiness Zone 6. The pitchers eventually die down after the prolonged rigors of winter weather, but new growth resumes in early to mid-Spring (April in Chapel Hill and Charlotte, N.C.). Typical of the S. rubra ssp. rubra complex, new growth (i.e., new leaves and short growth of the rhizome) continues strongly all summer, stopping in mid-Autumn (October in Chapel Hill and Charlotte, N.C.).

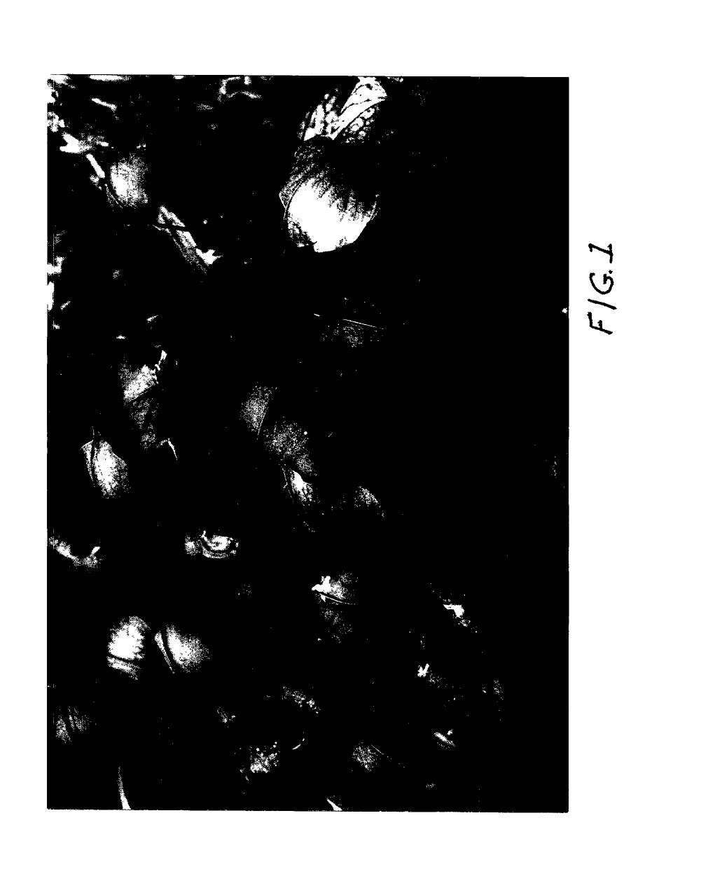

Vegetative Structures.—Sarracenia ‘Redbug’ is a clump-forming herbaceous perennial, growing from a horizontal rhizome that is typically approximately 5 mm in diameter and up to several centimeters long. Typically, thick white roots grow from the base of newer leaves. A cluster of tubular pitcher leaves arises from the growing tip of the rhizome. The leaves, at maturity, curve slightly forward and range from 15-25 cm tall, with a diameter at the widest point of 1.8-2.8 cm. Sarracenia ‘Redbug’ has a wide pitcher for its length (FIG. 1). The pitcher averages a width-to-length ratio of 0. 1, which is approximately twice the width-to-length ratios of the parents. A hood covers and extends beyond the orifice of the pitcher, and often has a wavy or slightly scalloped margin. There are no long hairs on the under surface of the hood. The hood measures up to 4.0 cm wide and 4.0 cm long, which is approximately two-times the size of the hood for the parent species. The tip of the hood forms a prominent filament up to 2.0 mm long, usually held horizontally. The leaves are of thick texture, and finely pubescent (like the S. rubra ssp. wherryi parent) on the outside. The rim of the orifice (pitcher mouth) is thick and revolute. There is a typical wing (flattened flange of tissue) along the front side of the pitcher along its entire length, up to 0.5 cm wide that is the same color as the tube. Furthermore, there are no winter phyllodia (non-pitcher leaves) produced by ‘Redbug’.

Pitcher Leaf Coloration.—FIG. 1 shows the distinctive coloration of a typical ‘Redbug’ specimen in the summer and in full sun. The rim of the orifice is dark Chrysanthemum Crimson (H.C.C. #860/2, page 175) on the front portion and green on the back portion (the S. rubra ssp. rubra parent is red-brown all around; the S. rubra ssp. wherryi parent is green all around). The upper three-quarters of a mature pitcher tube is Chrysanthemum Crimson (H.C.C. #824/2, page 169) and the lower quarter is Scheeles Green (H.C.C. #860/2, page 175). The veins on the upper pitcher form a distinct reticulate pattern on the outside and are seen as a deep cast of Chrysanthemum Crimson (H.C.C. #860/2, page 175).

Variations in Color.—In full sunlight conditions, the pitcher color starts out as a green suffused with a light cast of Chrysanthemum Crimson (H.C.C. #860/2, page 175) in the immature pitcher which develops into a deeper cast of Chrysanthemum Crimson (H.C.C. #860/2, page 175) in the mature pitcher. Similar to other species of Sarracenia, ‘Redbug’ will not robustly develop distinct pitchers under light intensity conditions of 50% or less, and the leaves typically remain green suffused with a light cast of Chrysanthemum Crimson (H.C.C. #824/2, page 169). In the summer, the upper three-quarters of the pitcher tube is Chrysanthemum Crimson (H.C.C. #824/2, page 169) and the lower quarter of the pitcher is Scheeles Green (H.C.C. #860/2, page 175) (FIG. 1). In the winter, the entire pitcher retains a Garnet Brown color (H.C.C. #00918/1, page 192).

Reproductive Structures.—In the late spring (late April to mid-May in Chapel Hill and Charlotte, N.C.) solitary, nodding flowers develop on a scape. A single flower has 5 sepals, 5 petals, many stamens, and 5 stigmas on an expanded umbrella-like style. Flower petals are Rose Bengal (H.C.C. #25/3, page 25), and there may be several produced from each growing point. The flowers are typical of other members of the S. rubra ssp. rubra complex (which includes both parents). Pollination results in numerous (up to 500) seeds produced in a capsule.

Comparison with Other Sarracenia Cultivars.—Sarracenia ‘Redbug’ differs from most other species of Sarracenia in that it is more compact (maximum leaf height of 25 cm compared to 91-122 cm for other species) and is more vigorous in its growth (producing multiple flushes of pitchers in a single growing season compared to a single flush produced by other species). Whereas the pitcher of S. rubra ssp. rubra typically has more brown coloration and S. rubra ssp. wherryi typically is a dull green with red veins, the pitcher of Sarracenia ‘Redbug’ is a deep cast of Chrysanthemum Crimson (H.C.C. #860/2, page 175). A typical ‘Redbug’ plant will, like the S. rubra ssp. wherryi parent, produce continuous flushes of leaves in a single summer season versus other pitcher plants which typically produce a single flush in a season. It differs from its S. rubra subspecies rubra parent in many aspects (see TABLE 1), including: 1) it has a distinctly fatter tube, particularly towards its apex (up to 2.5 cm in diameter verses 1.25 cm in diameter), 2) its hood is three times wider, 3) it has a more clumped appearance, and 4) it has a distinct coating of short fine hairs on the outer surface of the leaf giving it a ‘fuzzy’ feel and appearance. In addition to color differences, it may be distinguished from its other parent, S. rubra ssp. wherryi, in having a hood that is more than 1.5 times wider and a tube that is two times wider (TABLE 1). ‘Redbug’ also has a prominent filament formed at the tip of the hood, typically held horizontally, whereas in the S. rubra ssp. wherryi parent the filament is generally reflexed and held in a vertical position, and the filament is only very small or even absent in the S. rubra ssp. rubra parent. The flowers do not differ from any of the forms of S. rubra ssp. rubra or S. rubra ssp. wherryi.

Sarracenia ‘Redbug’ differs from all other hybrids in its combination of color, stature, texture, pitcher and hood shape, vigor, and winter appearance.

TABLE 1

| | S. rubra ssp. | S. rubra ssp. | |
|---|---|---|---|
| Characteristic | rubra | wherryi | Sarracenia ‘Redbug’ |
| Leaf Length | 140 mm | 156 mm | 190 mm |
| Diameter of Leaf | 8.2 mm | 9.0 mm | 20.1 mm |
| at Widest Point* | | | |
| Leaf Width to | 0.059 | 0.058 | 0.106 |
| Leaf Length Ratio | | | |
| Hood Width | 11.2 mm | 17.3 mm | 28.3 mm |
| Hood Filament | minuscule to | 1-2 mm reflexed, | 1-2 mm prominent, |
| (Tip) | absent | typically vertical | typically horizontal |

All data are averaged from 10 random measurements collected in December 2000 at the North Carolina Botanical Garden where the plants were grown in full sunlight outdoors.
*not to be confused with orifice diameter.

What is claimed is:

1. A new and distinct cultivar of Sarracenia plant named ‘Redbug’, substantially as illustrated and described herein.
